(19) United States
(12) Patent Application Publication
(10) Pub. No.: US 2023/0024614 A1
(43) Pub. Date: Jan. 26, 2023

(54) ENZYME COMPOSITIONS AND METHODS OF MAKING THEM

(71) Applicant: CLARA FOODS CO., SOUTH SAN FRANCISCO, CA (US)

(72) Inventors: Frank Douglas Ivey, South San Francisco, CA (US); Joel Andrew Kreps, South San Francisco, CA (US); Harshal Kshirsagar, South San Francisco, CA (US)

(21) Appl. No.: 17/808,260

(22) Filed: Jun. 22, 2022

Related U.S. Application Data
(63) Continuation of application No. PCT/US20/66729, filed on Dec. 22, 2020.
(60) Provisional application No. 62/953,361, filed on Dec. 24, 2019.

(51) Int. Cl. C12N 9/36; A23L 3/3571; C12N 15/81

(52) U.S. Cl. CPC C12N 9/2462; A23L 3/3571; C12Y 302/01017; C12N 15/815; A23V 2002/00

(57) ABSTRACT

Provided herein are compositions with enzymatically active enzymes produced recombinantly, enhanced protein content and methods for the preparation thereof.

### CROSS-REFERENCE TO RELATED APPLICATIONS

This application is a continuation application of International Patent Application PCT/US2020/066729, filed Dec. 22, 2020, which claims priority to U.S. Provisional Patent Application Ser. No. 62/953,361, filed Dec. 24, 2019, each of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Dec. 22, 2020, is named 49160_718_301_SL.txt and is 6,975 bytes in size.

### BACKGROUND

Proteins are important dietary nutrients. They can serve as a fuel sources and/or as sources of amino acids, including the essential amino acids that cannot be synthesized by the body. The daily recommended intake of protein for healthy human adults is 10% to 35% of a person's total calorie needs, and currently the majority of protein intake for most humans is from animal-based sources. In addition, athletes and bodybuilders may rely upon supplemental protein consumption to build muscle mass and improve performance. Recombinantly produced proteins are free from animal-based sources and provide an alternative protein resource for consumers. With the world population growing, and the coincidental increase in global food demand, there is an unmet need for alternative sustainable, non-animal-based sources of dietary and supplemental protein.

### SUMMARY

The methods and compositions of the present disclosure provide this unmet need.

In some embodiments, described herein are consumable compositions comprising a recombinant food preserving enzyme (rFPE). In some cases, the FPE may be a goose-type FPE (gFPE). In some cases, the composition may be semi-solid or a gel composition.

In some embodiments, the consumable composition may be free of bacterial impurities.

In some embodiments, the gFPE comprises an amino acid sequence with at least 95% identity to SEQ ID NO: 1. In some embodiments, the gFPE comprises an amino acid sequence with at least 97% identity to SEQ ID NO: 1.

In some embodiments, the gFPE comprises an amino acid sequence with at least 95% identity to SEQ ID NO: 2. In some embodiments, the gFPE comprises an amino acid sequence with at least 97% identity to SEQ ID NO: 2.

In some embodiments, the gFPE comprises an amino acid sequence with at least 95% identity to SEQ ID NO: 3. In some embodiments, the gFPE comprises an amino acid sequence with at least 97% identity to SEQ ID NO: 3.

In some embodiments, the gFPE comprises an amino acid sequence with at least 95% identity to SEQ ID NO: 4. In some embodiments, the gFPE comprises an amino acid sequence with at least 97% identity to SEQ ID NO: 4.

In some embodiments, the consumable composition may be heat treated. In some embodiments, the consumable composition has a longer shelf life than a nearly identical consumable composition which does not comprise the gFPE. In some embodiments, the consumable composition has a longer shelf life than the shelf life of a nearly identical consumable composition which comprises chicken egg-white muramidase rather than the gFPE. In some embodiments, the gFPE may be produced in a Pichia cell.

In some embodiments, described herein are compositions comprising a recombinant foodstuff preserving enzyme (FPE) wherein the FPE may have an activity of greater than 90,000 shugar U/mg.

In some cases, the FPE may have an activity in Shugar units of greater than 150,000 U/mg. The FPE may have an activity in Shugar units of greater than 200,000 U/mg. The FPE may have an activity in Shugar units of greater than 250,000 U/mg. The FPE may have an activity in Shugar units of greater than 300,000 U/mg. The FPE may have an activity in Shugar units of greater than 350,000 U/mg. The FPE may have an activity in Shugar units of greater than 400,000 U/mg. The FPE may have an activity in Shugar units of greater than 450,000 U/mg.

The recombinant FPE may be produced in a Pichia cell.

The composition may be a food composition. The food composition may comprise one or more consumable ingredients. The food composition may have a longer shelf life than a nearly identical food composition which does not comprise the recombinant FPE. The food composition may have a longer shelf life than the shelf life of a nearly identical product which may comprise chicken egg-white muramidase rather than the recombinant FPE.

The composition may be a powder composition comprising rFPE.

The recombinant FPE may comprise an amino acid sequences with at least 85% sequence identity to SEQ ID NO: 1.

One Shugar unit may be an amount of the enzyme which will digest a suspension of M. luteus cells causing a decrease in absorbance of the solution at a rate of 0.001 per minute at 37° C., pH 7.0.

The composition may be hypoallergenic as compared to a composition comprising chicken egg-white muramidase.

The recombinant FPE may have a comparable activity as compared to a non-recombinant FPE and/or a FPE comprising at least 85% sequence identity to SEQ ID NO: 1 yet may be isolated from a natural source.

In some embodiments, described herein is a consumable composition comprising a recombinant foodstuff preserving enzyme (FPE), wherein the FPE may comprise an amino acid sequence with at least 85% sequence identity to SEQ ID NO: 1.

The consumable composition may be a food composition. The food composition may have a gel-like texture or consistently. The food composition may be in the form of a baked product. The food composition may be in the form of an egg-white-like product. The FPE may be recombinantly produced in Pichia pastoris cells. The food composition may be in liquid form. The food composition may be in solid form.

The composition may have a longer shelf life than the shelf life of a nearly identical product which may comprise chicken egg-white muramidase rather than the recombinant FPE.

The food composition may have at least 0.1% FPE by weight. The food composition may have at most 10% FPE by weight. The FPE may be enzymatically active in the food composition. The composition may be an ingredient. The food composition may be substantially free of microorganisms or cell-debris. The food composition may be a probiotic formulation. The recombinant FPE may be at least 95% pure.

The food composition may comprise one or more recombinant proteins in addition to the recombinant FPE. The recombinant FPE may provide gel solidity or increased viscosity to the food product. The food composition may comprise more than one recombinant proteins in addition to the recombinant FPE.

In some embodiments, described herein are methods of preparing a consumable composition comprising steps of: providing an isolated foodstuff preserving enzyme (FPE) which is recombinantly produced and combining the recombinantly produced FPE with one or more consumable ingredients. In some cases, the FPE is a goose-type FPE.

The recombinantly produced FPE may have an amino acid sequence with at least 95% sequence identity to SEQ ID NO: 1.

The recombinantly produced FPE may be recombinantly produced in a yeast cell. The yeast cell may be Pichia pastoris.

The recombinantly produced FPE increases the shelf life of the consumable composition relative to a nearly identical consumable composition lacking the recombinantly produced FPE.

The recombinantly produced FPE provides a gel-like texture to the consumable composition.

The consumable composition may be a food product that may be ready for consumption by a human/animal.

In some embodiments, described herein are methods of producing a xanthan gum product comprising the steps of: providing X. campestris cells into a fermentation medium; heat treating the cells at a temperature between 45-60° C. and a pH of between 8-10 and an alkaline protease, thereby producing a solution comprising cell debris; adding to a solution comprising cell debris a foodstuff preserving enzyme (FPE) that may be recombinantly produced and/or a recombinantly produced FPE comprising an amino acid sequence with at least 95% sequence identity to SEQ ID NO: 1, thereby producing a xanthan gum solution. The method may also comprise adding isopropanol to the xanthan gum solution, thereby precipitating xanthan gum; isolating and drying the precipitated xanthan gum, thereby obtaining a xanthan gum product.

The amount of the FPE added may be less than the amount of chicken egg-white FPE that would be needed to produce an equivalent amount of the xanthan gum product under otherwise identical conditions.

The method of producing xanthan gum may further comprise a step of adjusting the pH of the composition after producing a solution comprising cell debris.

In some embodiments, described herein are food preservatives comprising a recombinantly produced food preserving enzyme (FPE) with at least 95% sequence identity to SEQ ID NO: 1.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1A to FIG. 1E illustrate bactericidal activity of a recombinant foodstuff preserving enzyme (rFPE) against common food spoiling bacteria.

FIG. 2A to FIG. 2E illustrate gelation of an rFPE at various temperatures and concentrations.

FIG. 3 illustrates the lack of reactivity of rFPE1an anti-chicken muramidase antibody to an rFPE.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure relates to recombinant foodstuff preserving enzymes (rFPEs) that provide superior properties relative to current commercially available enzyme products. In the food industries, agents are added to a food product to reduce spoilage by microbes. Common examples of antimicrobial food preservatives include compounds such as sodium benzoate, benzoic acid, nitrites, sulfites, sodium sorbate and potassium sorbate. Alternately, enzymes that possess antimicrobial activity may be added to a food product; an advantage of which is that the enzymes provide both antimicrobial activity and increase the protein content of the food product. The present disclosure relates to rFPE which possess antimicrobial activity, increase the protein content, and, further, provide favorable qualities to the food product, e.g., increased gelling and firmness to a solid or semi-solid food product or increased viscosity to a liquid food product. As disclosed herein, the rFPEs of the present disclosure demonstrate unexpectedly superior qualities relative to commercially available enzymes, which are used for their antimicrobial activity in food products.

Provided herein are consumable compositions comprising a foodstuff preserving enzyme (FPE). Such consumable compositions can be used in a food product, drink product, nutraceutical, pharmaceutical, cosmetic, or as an ingredient for a final product. In many embodiments herein, the consumable composition is in a liquid form or a semi-solid form (e.g., a gel). Preferably, the FPE in such compositions is made recombinantly, and may be referred to herein as a recombinant FPE (rFPE).

Unless indicated otherwise, the term FPE includes both FPE and rFPE. The FPE or rFPE in the consumable compositions herein is provided in concentrations that both increase the protein content of the consumable composition and also maintain one or more additional characteristics such as high clarity, reduced turbidity, or substantial sensory neutrality.

The use of rFPE in any of the consumable compositions herein allows for a non-animal-based source of protein, while maintaining a consumer-favorable sensory profile. Various embodiments of such compositions, methods of making them, and methods of using them are provided herein.

Provided herein are some exemplary embodiments of the disclosure. In one instance, a foodstuff preserving enzyme (FPE) such as a g-type FPE (gFPE) may be recombinantly produced in a host cell. The host cell may be a bacterial or yeast, or another fungal host cell. gFPE may be secreted by the host cell and collected and purified from the culture media. The purified gFPE may be lyophilized and used as an ingredient in a consumable composition. In some cases, an end user may be provided a lyophilized powder composition comprising primarily of gFPE protein. The end user may use gFPE as an ingredient in food compositions. In one instance, the user can produce a gel comprising gFPE by heat treatment at temperatures ranging from 50° C. to 120° C. The gel may be produced with or without the intention of using the FPE as a digestive enzyme as an antimicrobial. For instance, as described herein, gFPE has the unexpected effect of forming a gel in a solution without needing additional gelling agents. A user therefore may be able to gel a gFPE-comprising composition solely by heat treatment.

In another instance, a user may use gFPE as one ingredient in a consumable composition comprising other gelling agents such as plant fibers, other proteins, binding agents, etc.

Food products/consumable compositions may comprise as little as 0.05% gFPE w/w or as high as 30% gFPE w/w. The lower amounts may increase the viscosity of a liquid consumable composition whereas the higher amount may transform a consumable composition into a solid or semi-solid state.

In another instance, a user may use gFPE to degrade microbial cell walls to form a gum like substance such as Xanthan gum. The xanthan gum may then be added to a food composition.

In one example, a rFPE with a SEQ ID NO: 1 (rFPE1) may be produced recombinantly in a yeast host cell. rFPE1 may be secreted by the host cell and collected and isolated from the culture broth. It may also be purified and lyophilized. rFPE1 may then be used to provide a function such as a longer shelf life due to its anti-microbial activities or gelation due to its low thermal gelation profile in a consumable composition. rFPE1 may increase the nutritional content of a composition in addition to providing functional properties.

### Food-Preserving Enzymes

The FPE may include enzymes that are able to effectively hydrolyze the peptidoglycan of the bacterial cell wall by cleaving the β-1,4-glycosidic bond between the N-acetylmuramic acid and the N-acetylglucosamine of the peptidoglycan. FPEs in some cases may be a muramidase. FPEs in some cases may be a lysozyme. FPEs in some cases may be a goose-type lysozyme and may be referred to as “gFPE”.

FPE in some cases may be an enzyme with the amino acid sequence of SEQ ID NO: 1 or an enzymatically active fragment thereof or an enzyme with an amino acid sequence with at least 95% sequence identity to SEQ ID NO: 1. A FPE may be an enzyme with the amino acid sequence of SEQ ID NO: 2 or an enzymatically active fragment thereof or an enzyme with an amino acid sequence with at least 95% sequence identity to SEQ ID NO: 2. A FPE may be an enzyme with the amino acid sequence of SEQ ID NO: 3 or an enzymatically active fragment thereof or an enzyme with an amino acid sequence with at least 95% sequence identity to SEQ ID NO: 3. A FPE may be an enzyme with the amino acid sequence of SEQ ID NO: 4 or an enzymatically active fragment thereof or an enzyme with an amino acid sequence with at least 95% sequence identity to SEQ ID NO: 4. An rFPE, e.g., “a rFPE described herein”, having any one of SEQ ID NO: 1 to SEQ ID NO: 4 or an enzymatically active fragment thereof or an enzyme with an amino acid sequence with at least 95% sequence identity to any one of SEQ ID NO: 1 to SEQ ID NO: 4 may be referred to herein as a gFPE.

The specific enzyme activity of a recombinant FPE such as a gFPE may be higher than the specific enzyme activity of a native muramidase isolated from natural sources (i.e., a non-recombinant muramidase) or a chicken muramidase. The specific enzymatic activity of the FPE may be measured using conventional assays such as the Shugar assay which measures the loss in absorbance of a solution comprising a microorganism such as Micrococcus lysodeikticus. One Shugar unit may be defined as the amount of enzyme that will disrupt the structural integrity of the cell walls in a solution of M. lysodeikticus, thus, causing a decrease in absorbancy of 0.0001 per minute at 25° C.

The specific enzymatic activity of an rFPE described herein, for instance a gFPE, may be at least 35,000 Shugar units/mg (U/mg). The specific activity of rFPEs such as a gFPE may be at least 50,000 Shugar U/mg, 60,000 Shugar U/mg, 70,000 Shugar U/mg, 80,000 Shugar U/mg, 90,000 Shugar U/mg, 100,000 Shugar U/mg, 110,000 Shugar U/mg, 120,000 Shugar U/mg, 130,000 Shugar U/mg, 140,000 Shugar U/mg, 150,000 Shugar U/mg, 170,000 Shugar U/mg, 200,000 Shugar U/mg, 220,000 Shugar U/mg, 250,000 Shugar U/mg, 270,000 Shugar U/mg, 300,000 Shugar U/mg, 350,000 Shugar U/mg, 400,000 Shugar U/mg, 450,000 Shugar U/mg, 500,000 Shugar U/mg, 550,000 Shugar U/mg, 600,000 Shugar U/mg, or 700,000 Shugar U/mg.

The specific enzymatic activity of rFPEs such as a gFPE may be 35,000 U/mg to 600,000 U/mg of protein. The specific activity of rFPEs such as a gFPE may be 35,000 U/mg to 50,000 U/mg, 35,000 U/mg to 75,000 U/mg, 35,000 U/mg to 100,000 U/mg, 35,000 U/mg to 150,000 U/mg, 35,000 U/mg to 175,000 U/mg, 35,000 U/mg to 200,000 U/mg, 35,000 U/mg to 250,000 U/mg, 35,000 U/mg to 300,000 U/mg, 35,000 U/mg to 500,000 U/mg, 35,000 U/mg to 600,000 U/mg, 50,000 U/mg to 75,000 U/mg, 50,000 U/mg to 100,000 U/mg, 50,000 U/mg to 150,000 U/mg, 50,000 U/mg to 175,000 U/mg, 50,000 U/mg to 200,000 U/mg, 50,000 U/mg to 250,000 U/mg, 50,000 U/mg to 300,000 U/mg, 50,000 U/mg to 500,000 U/mg, 50,000 U/mg to 600,000 U/mg, 75,000 U/mg to 100,000 U/mg, 75,000 U/mg to 150,000 U/mg, 75,000 U/mg to 175,000 U/mg, 75,000 U/mg to 200,000 U/mg, 75,000 U/mg to 250,000 U/mg, 75,000 U/mg to 300,000 U/mg, 75,000 U/mg to 500,000 U/mg, 75,000 U/mg to 600,000 U/mg, 100,000 U/mg to 150,000 U/mg, 100,000 U/mg to 175,000 U/mg, 100,000 U/mg to 200,000 U/mg, 100,000 U/mg to 250,000 U/mg, 100,000 U/mg to 300,000 U/mg, 100,000 U/mg to 500,000 U/mg, 100,000 U/mg to 600,000 U/mg, 150,000 U/mg to 175,000 U/mg, 150,000 U/mg to 200,000 U/mg, 150,000 U/mg to 250,000 U/mg, 150,000 U/mg to 300,000 U/mg, 150,000 U/mg to 500,000 U/mg, 150,000 U/mg to 600,000 U/mg, 175,000 U/mg to 200,000 U/mg, 175,000 U/mg to 250,000 U/mg, 175,000 U/mg to 300,000 U/mg, 175,000 U/mg to 500,000 U/mg, 175,000 U/mg to 600,000 U/mg, 200,000 U/mg to 250,000 U/mg, 200,000 U/mg to 300,000 U/mg, 200,000 U/mg to 500,000 U/mg, 200,000 U/mg to 600,000 U/mg, 250,000 U/mg to 300,000 U/mg, 250,000 U/mg to 500,000 U/mg, 250,000 U/mg to 600,000 U/mg, 300,000 U/mg to 500,000 U/mg, 300,000 U/mg to 600,000 U/mg, or 500,000 U/mg to 600,000 U/mg of protein. The specific activity of rFPEs such as a gFPE may be at most 50,000 U/mg, 75,000 U/mg, 100,000 U/mg, 150,000 U/mg, 175,000 U/mg, 200,000 U/mg, 250,000 U/mg, 300,000 U/mg, 500,000 U/mg, or 600,000 U/mg of protein.

The specific activity of an rFPE such as a gFPE may be comparable to or more than the specific activity of a muramidase isolated from natural sources, such as egg whites. The specific activity of an rFPEs such as a gFPE may be comparable to or more than the specific activity of a chicken muramidase isolated from egg whites or produced recombinantly.

The rFPEs described herein, such as gFPE, may have antimicrobial activity. The antimicrobial activity of a FPE produced recombinantly such as gFPE/rFPE1 may be comparable to or higher than the antimicrobial activity of a commercially-available muramidase and/or a non-recombinant muramidase The antimicrobial activity of a gFPE may be comparable to or higher than the antimicrobial activity of a chicken muramidase.

Due to the high specific activity of gFPEs described herein, food products comprising gFPE may have a shelf-life comparable to or longer than the shelf-life of food products made without gFPE. The shelf-life of food products comprising gFPE may be comparable to or longer than the shelf-life of food products made using a commercially available muramidase and/or a non-recombinant muramidase.

The gFPEs described herein, when produced recombinantly, may be hypoallergenic as compared to a muramidase isolated from natural source. In some cases, rFPE1 may be hypoallergenic as compared to chicken muramidase.

### Consumable Compositions

Consumable compositions disclosed herein include products that comprise, consists essentially of, or consists of FPE, preferably rFPE or gFPE. Consumable compositions may comprise naturally isolated FPE1 or gFPE in addition to rFPE1.

A consumable composition disclosed herein can have a rFPE concentration of about 0.5% to about 25%. A consumable composition disclosed herein can have a rFPE concentration of about 0.5% to about 1%, about 0.5% to about 2%, about 0.5% to about 5%, about 0.5% to about 7%, about 0.5% to about 10%, about 0.5% to about 15%, about 0.5% to about 20%, about 0.5% to about 25%, about 1% to about 2%, about 1% to about 5%, about 1% to about 7%, about 1% to about 10%, about 1% to about 15%, about 1% to about 20%, about 1% to about 25%, about 2% to about 5%, about 2% to about 7%, about 2% to about 10%, about 2% to about 15%, about 2% to about 20%, about 2% to about 25%, about 5% to about 7%, about 5% to about 10%, about 5% to about 15%, about 5% to about 20%, about 5% to about 25%, about 7% to about 10%, about 7% to about 15%, about 7% to about 20%, about 7% to about 25%, about 10% to about 15%, about 10% to about 20%, about 10% to about 25%, about 15% to about 20%, about 15% to about 25%, or about 20% to about 25%. A consumable composition disclosed herein can have a rFPE concentration of about 0.5%, about 1%, about 2%, about 5%, about 7%, about 10%, about 15%, about 20%, or about 25%. A consumable composition disclosed herein can have a rFPE concentration of at least about 0.5%, about 1%, about 2%, about 5%, about 7%, about 10%, about 15%, or about 20%. A consumable composition disclosed herein can have a rFPE concentration of at most about 1%, about 2%, about 5%, about 7%, about 10%, about 15%, about 20%, or about 25%.

A consumable product can include one or more other proteins, such as a non-FPE protein or a non-recombinant protein. The rFPE can increase amount of protein content in a consumable product. For example, the consumable composition can include a whey protein, a pea protein, a soy protein, an almond protein, an oat protein, a flax seed protein, a vegetable protein, or an egg-white protein. In some cases, the one or more other proteins can comprise rFPE from avian, fish, amphibian, or reptile sources.

A consumable composition can be an ingredient of a final product or finished product. The FPE composition can be an ingredient that is then mixed with other ingredients to make a final product for an end-user. A final or finished product is one that is ready for an end-user's consumption or use. The finished product can be a processed product, such as processed food or a processed drink. Non-limiting example of consumable compositions include food products, beverage products, dietary supplements, food additives, nutraceuticals, healthcare products, and cosmetics.

The consumable compositions disclosed herein can be a liquid or a semi-solid. The consumable composition may have a gel-like texture. Any of the liquid or semi-solid consumable compositions disclosed herein can be created by mixing a powdered rFPE into a solution. The solution can be the final product or an intermediate solution which is then further modified to generate a final product.

Examples of liquid consumable compositions or beverages include: a soda, a vitamin drink, a protein shake, a meal replacement shake, a juice, a refreshment drink, a milk based drink or a non-dairy based drink, flavored water, a carbonated drink, coffee, caffeinated drink, tea, beer, liquor, and a sports drink. In liquid consumable compositions, the rFPE provides increased viscosity to the liquid composition.

Clarity can also be determined by lack of translucency. A material that lacks translucency may also have a milky, white or opaque appearance. Consumable compositions with rFPE may lack a milky, white or opaque appearance.

A consumable composition with rFPE may also have an improved sensory appeal as compared to the composition without rFPE or with a different enzyme present in an equal concentration to the rFPE.

As described herein, a consumable composition can be in a liquid form. A liquid form can be an intermediate product such as soluble rFPE solution. In some cases, a liquid form can be a final product, such as a beverage comprising rFPE. Example of different types of beverages contemplated herein include: a juice, a soda, a soft drink, a flavored water, a protein water, a fortified water, a carbonated water, a nutritional drink, an energy drink, a sports drink, a recovery drink, a heated drink, a coffee-based drink, a tea-based drink, a plant-based milk, a milk based drink, a non-dairy, plant based milk drink, infant formula drink, an alcoholic drink and a meal replacement drink.

In some embodiments, the consumable food composition may be in a semi-solid form. The food product can be a jelly, a candy, a broth, a soup, a gelatin-containing product, a gelled product and a gummy product. Additional exemplary categories of food products in which rFPE can be added include sauces, dressings, and condiments.

In some embodiments, the consumable food composition may be in a solid form. The composition may be a baked good, a bread, a gluten containing product, a gluten free product, a sauce, a dressing, a condiment, a spice blend, a seasoning mix, a coating, a breading, a fruit snack, a vegetable snack, a frozen dairy product, a frozen “dairy-like” product, a prepared meal, a meat product, a meatless product, a burger, a patty, a protein supplement, a nutrition bar, a dessert, or an “egg-like” product.

In some embodiments, the consumable food compositions and methods of making such compositions include a heating condition. For example, a consumable food composition may be a heated or hot beverage, such as a warm or hot drink, a soup or a broth. In some cases, a consumable food composition may have a heating step for producing an ingredient or a finished product. Other examples include pan frying and baking.

In some embodiments herein, a consumable food composition containing gFPE is a composition that is used as an ingredient with other ingredient(s) or component(s) to create a finished product. For example, gFPE can be mixed with water or other liquid, and then this mixture used as an ingredient to create a beverage, food product, dietary supplement or nutraceutical. In some cases, gFPE is mixed with other ingredients, such as other liquids (e.g., nut milks, fruit juices, vegetable extracts or carbonated solutions. This solution can be an ingredient that is then mixed with other ingredients to make a final product for an end-user; for example, the solution may be a syrup containing concentrated gFPE. A final or finished product is one that is ready for an end-user's consumption. The finished product can be a processed product, such as processed food or a processed drink. In some instances, the gFPE is provided in a separate container to be mixed into the final product by the end-user. In some cases, gFPE is mixed with other ingredients, such as gelling agents to make candies, gummy products, gelled products (such as a Jello™) or sports gels.

During or after preparation of a consumable food product containing gFPE may be formulated as a liquid, solid, syrup, or powder. A composition may be refrigerated, frozen, stored warm, stored at room temperature or held at a heated temperature. Preparation of the food product can include a heating-step or the food product is stored or served at a heated temperature.

Examples of liquid consumable compositions or beverages include: a soda, a vitamin drink, a protein shake, a meal replacement shake, a juice, a refreshment drink, a milk-based drink or a non-dairy based drink, flavored water, a carbonated drink, coffee, caffeinated drink, tea, flower-based drink, beer, liquor, and a sports drink.

Any of the liquid or semi-solid consumable compositions herein can be created by mixing a powdered gFPE into a solution. The solution can be the final product or an intermediate solution which is then further modified to generate a final product.

Examples of solvents that can be used to prepare an gFPE solution include still water, carbonated water, alcohol, juices, and any other commercially available drink including those described in more detail herein.

A method of generating a consumable composition comprising gFPE may comprise mixing gFPE with a solvent and, optionally, one or more other components. The mixing may be performed by any conventionally used mixing method including mortar and pestle, mechanical grinder, blending, homogenization process or a sonication process.

The amount of gFPE added to the solution can be one that generates an gFPE concentration as derived herein (either in the final product or an intermediate product).

Preferably, addition of the gFPE to the solution results in most or nearly all of the gFPE solubilized into the solution at room temperature. In one instance, solubility is determined based on clarity or degree of lack of turbidity.

The consumable compositions herein can also be subjected to a heating step. Such a step can modify or increase solubility of the gFPE. For example, it was found that performing a heating step in the process of making a product such as retorting, hot filling, or pasteurization can increase solubility and hence clarity of an gFPE solution herein.

Preparation of a consumable food product containing gFPE may include drying and/or concentrating. In some cases, drying forms a dry, dehydrated, concentrated, and/or solid protein or composition. Some non-limiting examples of drying methods include thermal drying, evaporation (e.g., by means of vacuum or air), distillation, boiling, heating in an oven, vacuum drying, spray drying, freeze drying, and lyophilization, or any combination thereof.

Preparation of a consumable food product containing gFPE may include diluting and/or hydrating. In some cases, the diluting may comprise addition of a liquid, which may be water or another liquid form. For example, a composition can be diluted (e.g., from 20% water to 99.9% water). In another example, a dry composition can be hydrated (e.g., from a dry solid to 99.9% water).

In some embodiments, the consumable food composition containing gFPE is in powder form and when the powdered composition is formulated into a solution, the gFPE is substantially fully soluble. In some embodiments, when the powdered composition is formulated into a solution, the gFPE is substantially fully soluble and the solution is substantially clear. In some embodiments, when the powdered composition is formulated into a solution, the gFPE is substantially fully soluble, the solution is substantially clear and the solution is essentially sensory neutral or has an improved sensory appeal as compared to solutions made with other powderized proteins such whey protein, soy protein, pea protein, egg white protein or whole egg proteins. In some embodiments, the powdered composition is solubilized in water where the concentration of gFPE is or is about 1%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% weight per total weight (w/w) and/or weight per total volume (w/v) of composition.

In some embodiments of the consumable food compositions described herein, the composition is essentially free of animal-derived component, whey protein, caseinate, fat, lactose, hydrolyzed lactose, soy protein, collagen, hydrolyzed collagen, or gelatin, or any combination thereof. A composition described herein may be essentially free of cholesterol, glucose, fat, saturated fat, trans fat, or any combination thereof. In some cases, a composition described herein comprises less than 10%, 5%, 4%, 3%, 2%, 1%, or 0.5% fat by dry weight. In some embodiments, the composition may be fat-containing (e.g., such as a mayonnaise) and such composition may include up to about 60% fat or a reduced-fat composition (e.g., reduced fat mayonnaise) and such composition may include lesser percentages of fat. A composition that free of an animal-derived component can be considered vegetarian and/or vegan.

In some embodiments, an gFPE powder composition comprises less than 5% ash. The term “ash” is an art-known term and represents inorganics such as one or more ions, elements, minerals, and/or compounds In some cases, the gFPE powder composition comprises less than 5%, 4.5%, 4%, 3.5%, 3%, 2.5%, 2%, 1.5%, 1%, 0.75%, 0.5%, 0.25% or 0.1% ash weight per total weight (w/w) and/or weight per total volume (w/v).

In some embodiments, the moisture content of an gFPE powder composition may be less than 15%. The gFPE powder composition may have less than 15%, 12%, 10%, 8%, 6%, 5%, 3%, 2% or 1% moisture weight per total weight (w/w) and/or weight per total volume (w/v). In some embodiments, the carbohydrate content of an gFPE powder composition may be less than 30%. The gFPE powder composition may have less than 30%, 27%, 25%, 22%, 20%, 17%, 15%, 12%, 10%, 8%, 5%, 3% or 1% carbohydrate content w/w or w/v.

In some cases, the protein content of an gFPE powder composition may be 30% to 99% weight per total weight (w/w) and/or weight per total volume (w/v). In some cases, the protein content of an gFPE powder composition may be at least 30% w/w or w/v. In some cases, the protein content of an gFPE powder composition may be at most 99% w/w or w/v. In some cases, the protein content of an gFPE powder composition may be 30% to 40%, 30% to 50%, 30% to 60%, 30% to 70%, 30% to 75%, 30% to 80%, 30% to 85%, 30% to 90%, 30% to 95%, 30% to 99%, 40% to 50%, 40% to 60%, 40% to 70%, 40% to 75%, 40% to 80%, 40% to 85%, 40% to 90%, 40% to 95%, 40% to 99%, 50% to 60%, 50% to 70%, 50% to 75%, 50% to 80%, 50% to 85%, 50% to 90%, 50% to 95%, 50% to 99%, 60% to 70%, 60% to 75%, 60% to 80%, 60% to 85%, 60% to 90%, 60% to 95%, 60% to 99%, 70% to 75%, 70% to 80%, 70% to 85%, 70% to 90%, 70% to 95%, 70% to 99%, 75% to 80%, 75% to 85%, 75% to 90%, 75% to 95%, 75% to 99%, 80% to 85%, 80% to 90%, 80% to 95%, 80% to 99%, 85% to 90%, 85% to 95%, 85% to 99%, 90% to 95%, 90% to 99%, or 95% to 99% w/w or w/v. In some cases, the protein content of an gFPE powder composition may be about 30%, 40%, 50%, 60%, 70%, 75%, 80%, 85%, 90%, 95%, or 99% w/w or w/v. In some cases, the protein content of an gFPE powder composition may be at least 30%, 40%, 50%, 60%, 70%, 75%, 80%, 85%, 90% or 95% w/w or w/v. In some cases, the protein content of an gFPE powder composition may be at most 40%, 50%, 60%, 70%, 75%, 80%, 85%, 90%, 95%, or 99% w/w or w/v.

Gelation of rFPEs

In some embodiments, FPEs described herein may form a semi-solid composition. In some cases, a semi-solid composition may be produced upon heat treatment of the FPEs described herein. gFPE may provide gelation to a food product. gFPEs may be used to degrade or digest cell wall peptidoglycans of certain microorganisms such as bacteria to form gels. In some cases, gFPE may be able to form a gel without degrading or digesting a microbial cell wall. In some cases, a gel composition formed upon heat treatment of gFPE may not comprise any microbial impurities. In some cases, a gel composition formed upon heat treatment of a FPE such as gFPE may not comprise any bacterial impurities. In some cases, a gel composition formed upon heat treatment of gFPE may not comprise any other gelling or binding agents. In some cases, gFPE may provide improved gelation to a composition as compared to the gelation provided by a chicken muramidase.

In some cases, gFPE forms a gel upon heat treatment from a temperature ranging from 50° C. to 130° C. In some cases, gFPE forms a gel upon heat treatment from a temperature ranging from 50° C. to 130° C. at a w/w protein concentration of as low as 0.05%. In some cases, gFPE forms a gel upon heat treatment from a temperature ranging from 50° C. to 130° C. at a w/w protein concentration of as low as 0.05% in the absence of any other gelling agents.

In some cases, gFPEs may form a gel upon heat treatment at a temperature of 50° C. to 130° C. In some cases, gFPEs may form a gel upon heat treatment at a temperature of at least 50° C. In some cases, gFPEs may form a gel upon heat treatment at a temperature of at most 130° C. In some cases, gFPEs may form a gel upon heat treatment at a temperature of 50° C. to 60° C., 50° C. to 70° C., 50° C. to 75° C., 50° C. to 80° C., 50° C. to 90° C., 50° C. to 95° C., 50° C. to 100° C., 50° C. to 110° C., 50° C. to 120° C., 50° C. to 130° C., 60° C. to 70° C., 60° C. to 75° C., 60° C. to 80° C., 60° C. to 90° C., 60° C. to 95° C., 60° C. to 100° C., 60° C. to 110° C., 60° C. to 120° C., 60° C. to 130° C., 70° C. to 75° C., 70° C. to 80° C., 70° C. to 90° C., 70° C. to 95° C., 70° C. to 100° C., 70° C. to 110° C., 70° C. to 120° C., 70° C. to 130° C., 75° C. to 80° C., 75° C. to 90° C., 75° C. to 95° C., 75° C. to 100° C., 75° C. to 110° C., 75° C. to 120° C., 75° C. to 130° C., 80° C. to 90° C., 80° C. to 95° C., 80° C. to 100° C., 80° C. to 110° C., 80° C. to 120° C., 80° C. to 130° C., 90° C. to 95° C., 90° C. to 100° C., 90° C. to 110° C., 90° C. to 120° C., 90° C. to 130° C., 95° C. to 100° C., 95° C. to 110° C., 95° C. to 120° C., 95° C. to 130° C., 100° C. to 110° C., 100° C. to 120° C., 100° C. to 130° C., 110° C. to 120° C., 110° C. to 130° C., or 120° C. to 130° C. In some cases, gFPEs may form a gel upon heat treatment at a temperature of 50° C., 60° C., 70° C., 75° C., 80° C., 90° C., 95° C., 100° C., 110° C., 120° C., or 130° C. In some cases, gFPEs may form a gel upon heat treatment at a temperature of at least 50° C., 60° C., 70° C., 75° C., 80° C., 90° C., 95° C., 100° C., 110° C. or 120° C. In some cases, gFPEs may form a gel upon heat treatment at a temperature of at most 60° C., 70° C., 75° C., 80° C., 90° C., 95° C., 100° C., 110° C., 120° C., or 130° C.

In some cases, gFPE may form a gel at a concentration of 0.05% to 30% w/w. In some cases, gFPE may form a gel at a concentration of at least 0.05% w/w. In some cases, gFPE may form a gel at a concentration of at most 30% w/w. In some cases, gFPE may form a gel at a concentration of 0.05% to 1%, 0.05% to 2%, 0.05% to 5%, 0.05% to 8%, 0.05% to 10%, 0.05% to 15%, 0.05% to 20%, 0.05% to 25%, 0.05% to 30%, 1% to 2%, 1% to 5%, 1% to 8%, 1% to 10%, 1% to 15%, 1% to 20%, 1% to 25%, 1% to 30%, 2% to 5%, 2% to 8%, 2% to 10%, 2% to 15%, 2% to 20%, 2% to 25%, 2% to 30%, 5% to 8%, 5% to 10%, 5% to 15%, 5% to 20%, 5% to 25%, 5% to 30%, 8% to 10%, 8% to 15%, 8% to 20%, 8% to 25%, 8% to 30%, 10% to 15%, 10% to 20%, 10% to 25%, 10% to 30%, 15% to 20%, 15% to 25%, 15% to 30%, 20% to 25%, 20% to 30%, or 25% to 30% w/w. In some cases, gFPE may form a gel at a concentration of 0.05%, 1%, 2%, 5%, 8%, 10%, 15%, 20%, 25%, or 30% w/w. In some cases, gFPE may form a gel at a concentration of at least 0.05%, 1%, 2%, 5%, 8%, 10%, 15%, 20% or 25% w/w. In some cases, gFPE may form a gel at a concentration of at most 1%, 2%, 5%, 8%, 10%, 15%, 20%, 25%, or 30% w/w.

In some cases, gFPE may gel at low concentrations such as a concentration of 0.05% to 2% w/w. In some cases, gFPE may gel at a concentration of at least 0.05% w/w. In some cases, gFPE may gel at a concentration of at most 2% w/w. In some cases, gFPE may gel at a concentration of 0.05% to 0.06%, 0.05% to 0.125%, 0.05% to 0.25%, 0.05% to 0.5%, 0.05% to 1%, 0.05% to 1.5%, 0.05% to 2%, 0.06% to 0.125%, 0.06% to 0.25%, 0.06% to 0.5%, 0.06% to 1%, 0.06% to 1.5%, 0.06% to 2%, 0.125% to 0.25%, 0.125% to 0.5%, 0.125% to 1%, 0.125% to 1.5%, 0.125% to 2%, 0.25% to 0.5%, 0.25% to 1%, 0.25% to 1.5%, 0.25% to 2%, 0.5% to 1%, 0.5% to 1.5%, 0.5% to 2%, 1% to 1.5%, 1% to 2%, or 1.5% to 2% w/w. In some cases, gFPE may gel at a concentration of 0.05%, 0.06%, 0.125%, 0.25%, 0.5%, 1%, 1.5%, or 2% w/w. In some cases, gFPE may gel at a concentration of at least 0.05%, 0.06%, 0.125%, 0.25%, 0.5%, 1% or 1.5% w/w. In some cases, gFPE may gel at a concentration of at most 0.05%, 0.06%, 0.125%, 0.25%, 0.5%, 1%, 1.5%, or 2% w/w.

A semi-solid or gel consumable composition such as a foodstuff may comprise from 0.05% to 25% gFPE w/w. A semi-solid or gel consumable composition such as a foodstuff may comprise from at least 0.05% gFPE w/w. A semi-solid or gel consumable composition such as a foodstuff may comprise from at most 25% gFPE w/w. A semi-solid or gel consumable composition such as a foodstuff may comprise from 0.05% to 0.1%, 0.05% to 1%, 0.05% to 2%, 0.05% to 5%, 0.05% to 10%, 0.05% to 15%, 0.05% to 20%, 0.05% to 25%, 0.1% to 1%, 0.1% to 2%, 0.1% to 5%, 0.1% to 10%, 0.1% to 15%, 0.1% to 20%, 0.1% to 25%, 1% to 2%, 1% to 5%, 1% to 10%, 1% to 15%, 1% to 20%, 1% to 25%, 2% to 5%, 2% to 10%, 2% to 15%, 2% to 20%, 2% to 25%, 5% to 10%, 5% to 15%, 5% to 20%, 5% to 25%, 10% to 15%, 10% to 20%, 10% to 25%, 15% to 20%, 15% to 25%, or 20% to 25% gFPE w/w. A semi-solid or gel consumable composition such as a foodstuff may comprise from 0.05%, 0.1%, 1%, 2%, 5%, 10%, 15%, 20%, or 25% gFPE w/w.

A composition comprising gFPEs may have a gel strength greater than a gel strength of c-type lysozymes or a chicken muramidase containing composition. In some cases, a gFPE composition has a gel strength of about or at least 50%, 55%, 60%, 65%, 70%, 75%, 80%, 85%, 90%, 95%, 100% relative to a chicken muramidase, or a c-type lysozyme containing composition. In some cases, an gFPE composition has a gel strength of up to 50%, 55%, 60%, 65%, 70%, 75%, 80%, 85%, 90%, 95%, 100% relative to a chicken muramidase, or a c-type lysozyme containing composition.

A liquid composition comprising gFPEs may have a viscosity greater than the viscosity of a composition comprising c-type lysozymes or a chicken muramidase. In some cases, a liquid gFPE composition has a viscosity of about or at least 50%, 55%, 60%, 65%, 70%, 75%, 80%, 85%, 90%, 95%, 100% relative to a chicken muramidase, or a c-type lysozyme containing composition. In some cases, a liquid gFPE composition has a viscosity of up to 50%, 55%, 60%, 65%, 70%, 75%, 80%, 85%, 90%, 95%, 100% relative to a chicken muramidase, or a c-type lysozyme containing composition.

Use of gFPE/FPE1 in Gum Production

gFPEs may be used to produce clear, vegan gels and as gums used as ingredients in food products. For instance, gFPE may be used to make bacterial polysaccharides such as xanthan gum, gellan or diutan gums. The transmittance of a gum containing solution produced using gFPE may be higher than the transmittance of a gum produced using conventionally used muramidases such as chicken muramidase thus leading to increased clarity in a gel formed using gFPE. The clarity of a gum containing solution produced using gFPE may be higher than the clarity of a gum produced using conventionally used muramidases such as chicken muramidase.

As the activity of rFPEs described herein are unexpectedly high, the production of gums and gels described herein may require less enzyme than is required when using a muramidase isolated from natural sources or chicken muramidase. In some cases, the amount or enzyme units required for the production of a gum or gel may be 2 times, 3 times, 5 times, 7 times or 10 times less than the amount or enzyme units required for the production of an identical a gum or gel using naturally obtained muramidases such as chicken muramidase.

Gums and gels as described herein may be produced by digesting bacterial cultures. Post-fermentation, the bacterial cultures may be treated with alkaline proteases under high temperature and alkaline conditions. The bacteria may be cultured first at room temperature or a temperature between 25° C. and 37° C. For gum production, the temperature of the bacterial culture may be increased to about 45° C., about 47° C., about 50° C., about 52° C., about 55° C., about 57° C., about 60° C., about 65° C. or about 75° C.

The bacterial culture may also be treated with an alkaline protease with high pH. The bacterial culture may be treated with an alkaline protease at a pH of about 8, about 8.5, about 9, about 9.5, about 10, about 10.5 or about 11. The proteolysis of the bacterial culture using an alkaline protease may be followed by the treatment with FPEs such as gFPE.

The pH of the culture may be modified before the treatment with gFPE. gFPE treatment may be performed at a neutral pH. gFPE treatment may be performed at a pH of about 5.5, about 6, about 6.5, about 7, about 7.5 or about 8.

Temperature of the culture may also be modified before treatment with gFPE. gFPE treatment may be performed at a temperature of about 30° C., about 32° C., about 35° C., about 37° C. or about 40° C.

The reaction of gFPE with the culture to produce a gum, such as xanthan gum, may be terminated using alcohol. Isopropanol may be used to terminate the reaction of gFPE with the bacterial culture. Other alcohols or solutions that can terminate the reaction are also envisioned. The gum produced from such a reaction may then be extracted, e.g., precipitated, from the culture.

The gums produced from such reactions may be further treated before consumption. For instance, the gum may be heat treated or dried before consumption.

### Packaging

One of the benefits of the consumable compositions disclosed herein is that they allow for simpler packaging. In one instance, a consumable composition may be packaged in a clear container as the lack of turbidity in the composition results in a more consumer appealing product.

A consumable composition can be refrigerated, frozen, stored warm, stored at room temperature or held at a heated temperature.

### Recombinant FPE

rFPE can have an amino acid sequence from any species. For example, an rFPE can have an amino acid sequence of FPE from a bird, a fish, an amphibian, or a reptile. An rFPE having an amino acid sequence from an avian can be selected from the group consisting of: poultry, fowl, waterfowl, game bird, chicken, quail, turkey, duck, ostrich, goose, gull, guineafowl, pheasant, emu, and any combination thereof. An rFPE can have an amino acid sequence derived from a single species, such as Anser anser anser or Gallus gallus domesticus. Alternatively, an rFPE can have an amino acid sequence derived from two or more species, and as such be a hybrid.

An rFPE can be a non-naturally occurring variant of an FPE. Such variant can comprise one or more amino acid insertions, deletions, or substitutions relative to a native FPE sequence.

Such a variant can have at least 70%, 75%, 80%, 85%, 90%, 95%, 96%, 97%, 98%, or 99% sequence identity to any one of SEQ ID NOs: 1-4. Preferably, a variant can have at least 90% or higher sequence identity to any one of SEQ ID NOs: 1-4. In some cases, a preferred variant may have at least 95% sequence identity to any one of SEQ ID NOs: 1-4. In some cases, a preferred variant may have at least 97% sequence identity to any one of SEQ ID NOs: 1-4. The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

In some embodiments, a variant is one that confers additional features, such as reduced allergenicity.

Depending on the host organism used to express the rFPE, the rFPE can have a glycosylation, acetylation, or phosphorylation pattern different from wildtype FPE. For example, the rFPE may or may not be glycosylated, acetylated, or phosphorylated. An rFPE may have an avian, non-avian, microbial, non-microbial, mammalian, or non-mammalian glycosylation, acetylation, or phosphorylation pattern.

In some cases, rFPE may be deglycosylated (e.g., chemically, enzymatically, Endo-H, PNGase F, O-Glycosidase, Neuraminidase, β1-4 Galactosidase, β-N-acetylglucosaminidase), deacetylated (e.g., protein deacetylase, histone deacetylase, sirtuin), or dephosphorylated (e.g., acid phosphatase, lambda protein phosphatase, calf intestinal phosphatase, alkaline phosphatase). Deglycosylation, deacetylation or dephosphorylation may produce a protein that is more uniform or is capable of producing a composition with less variation.

An rFPE is recombinantly expressed in a host cell. As used herein, a “host” or “host cell” denotes here any protein production host selected or genetically modified to produce a desired product. Exemplary hosts include fungi, such as filamentous fungi, as well as bacteria, yeast, plant, insect, and mammalian cells. A host cell may be Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum gloeosporiodes, Endothia spp., Endothia parasitica, Escherichia coli, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, or Trichoderma vireus. A host cell can be an organism that is approved as generally regarded as safe by the U.S. Food and Drug Administration.

An rFPE protein can be recombinantly expressed in yeast, filamentous fungi or a bacterium. In some embodiments, rFPE protein is recombinantly expressed in a Pichia species (Komagataella phaffii and Komagataella pastoris), a Saccharomyces species, a Trichoderma species, a Pseudomonas species or an E. coli species.

Expression of rFPE in a host cell, for instance, a Pichia species, a Saccharomyces species, a Trichoderma species, a Pseudomonas species may lead to an addition of peptides to the FPE sequence as part of post-transcriptional or post-translational modifications. Such peptides may not be part of the native FPE sequences. For instance, expressing a FPE sequence in a Pichia species, such as Komagataella phaffii and Komagataella pastoris may lead to addition of a peptide at the N-terminus or C-terminus. In some cases, a tetrapeptide EAEA is added to the N-terminus of the FPE sequence upon expression in a host cell.

Expression of an rFPE can be provided by an expression vector, a plasmid, a nucleic acid integrated into the host genome or other means. For example, a vector for expression can include: (a) a promoter element, (b) a signal peptide, (c) a heterologous FPE sequence, and (d) a terminator element.

Expression vectors that can be used for expression of FPE include those containing an expression cassette with elements (a), (b), (c) and (d). In some embodiments, the signal peptide (c) need not be included in the vector. In general, the expression cassette is designed to mediate the transcription of the transgene when integrated into the genome of a cognate host microorganism.

To aide in the amplification of the vector prior to transformation into the host microorganism, a replication origin (e) may be contained in the vector (such as PUC_ORIC and PUC (DNA2.0)). To aide in the selection of microorganism stably transformed with the expression vector, the vector may also include a selection marker (0 such as URA3 gene and Zeocin resistance gene (ZeoR). The expression vector may also contain a restriction enzyme site (g) that allows for linearization of the expression vector prior to transformation into the host microorganism to facilitate the expression vectors stable integration into the host genome. In some embodiments the expression vector may contain any subset of the elements (b), (e), (f), and (g), including none of elements (b), (e), (f), and (g). Other expression elements and vector element known to one of skill in the art can be used in combination or substituted for the elements described herein.

Exemplary promoter elements (a) may include, but are not limited to, a constitutive promoter, inducible promoter, and hybrid promoter. Promoters include, but are not limited to, acu-5, adh1+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbh1), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, inv1+, isocitrate lyase (ICL1), acetohydroxy acid isomeroreductase (ILV5), KAR2, KEX2, β-galactosidase (lac4), LEU2, melO, MET3, methanol oxidase (MOX), nmt1, NSP, pcbC, PET9, peroxin 8 (PEX8), phosphoglycerate kinase (PGK, PGK1), pho1, PHO5, PHO89, phosphatidylinositol synthase (PIS1), PYK1, pyruvate kinase (pki1), RPS7, sorbitol dehydrogenase (SDH), 3-phosphoserine aminotransferase (SER1), SSA4, SV40, TEF, translation elongation factor 1 alpha (TEF1), THI11, homoserine kinase (THR1), tpi, TPS1, triose phosphate isomerase (TPI1), XRP2, YPT1, and any combination thereof.

A signal peptide (b), also known as a signal sequence, targeting signal, localization signal, localization sequence, signal peptide, transit peptide, leader sequence, or leader peptide, may support secretion of a protein or polynucleotide. Extracellular secretion of a recombinant or heterologously expressed protein from a host cell may facilitate protein purification. A signal peptide may be derived from a precursor (e.g., prepropeptide, preprotein) of a protein. Signal peptides can be derived from a precursor of a protein other than the signal peptides in native FPE.

Any nucleic acid sequence that encodes FPE can be used as (c). Preferably such sequence is codon optimized for the host cell.

Exemplary transcriptional terminator elements include, but are not limited to, acu-5, adh1+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbh1), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, inv1+, isocitrate lyase (ICL1), acetohydroxy acid isomeroreductase (ILV5), KAR2, KEX2, β-galactosidase (lac4), LEU2, melO, MET3, methanol oxidase (MOX), nmt1, NSP, pcbC, PET9, peroxin 8 (PEX8), phosphoglycerate kinase (PGK, PGK1), pho1, PHO5, PHO89, phosphatidylinositol synthase (PIS1), PYK1, pyruvate kinase (pki1), RPS7, sorbitol dehydrogenase (SDH), 3-phosphoserine aminotransferase (SER1), SSA4, SV40, TEF, translation elongation factor 1 alpha (TEF1), THI11, homoserine kinase (THR1), tpi, TPS1, triose phosphate isomerase (TPI1), XRP2, YPT1, and any combination thereof.

Exemplary selectable markers (f) may include, but are not limited to: an antibiotic resistance gene (e.g. zeocin, ampicillin, blasticidin, kanamycin, nurseothricin, chloroamphenicol, tetracycline, triclosan, ganciclovir, and any combination thereof), an auxotrophic marker (e.g. ade1, arg4, his4, ura3, met2, and any combination thereof).

In one example, a vector for expression in Pichia sp. can include an AOX1 promoter operably linked to a signal peptide (alpha mating factor) that is fused in frame with a nucleic acid sequence encoding FPE, and a terminator element (AOX1 terminator) immediately downstream of the nucleic acid sequence encoding FPE.

In another example, a vector comprising a DAS1 promoter is operably linked to a signal peptide (alpha mating factor) that is fused in frame with a nucleic acid sequence encoding FPE and a terminator element (AOX1 terminator) immediately downstream of FPE.

A recombinant protein described herein may be secreted from the one or more host cells. In some embodiments, rFPE is secreted from the host cell. The secreted rFPE may be isolated and purified by methods such as centrifugation, fractionation, filtration, affinity purification and other methods for separating protein from cells, liquid and solid media components and other cellular products and byproducts. In some embodiments, rFPE is produced in a Pichia Sp. and secreted from the host cells into the culture media. The secreted rFPE is then separated from other media components for further use.

The consumable products and rFPE can be substantially free of any microbial growth. For instance, rFPE may be isolated from a culture comprising microbial growth. Alternatively, an rFPE composition may comprise microbial growth, for instance, in the case of probiotic formulations. In some cases, a probiotic composition comprises rFPE. A probiotic composition can comprise microbes that produce rFPE.

TABLE 1
Sequences

| | SEQ ID | |
|---|---|---|
| Identifier | NO. | Sequence |
| FPE1 | SEQ ID | RTDCYGNVNRIDTTGASCKTAKPEGLSYCGVSASKKIAERDLQAMDRYKTIIKKVGEKLCVEPAVIAGII |
| | NO: 1 | SRESHAGKVLKNGWGDRGNGFGLMQVDKRSHKPQGTWNGEVHITQGTTILINFIKTIQKKFPSWTKDQ |
| | | QLKGGISAYNAGAGNVRSYARMDIGTTHDDYANDVVARAQYYKQHGY* |
| Axolotl (g) | SEQ ID | SGCYGNIMDVPTTGASCLTASQDNLPYCGVAASQQMAATDLPDMNQYKEKILAVAQNLCMDGAVIA |
| (Ambystoma | NO: 2 | GIISRESRAGAVLQNGWGDNGHAFGLMQIDIRWHSIEGAWNSQENINEGTGILINMIVAISDKFPSWSVN |
| mexicanum) | | DNLKGGIAAYNAGPGNIYSYSQVDQYTTDGDYSNDVVARAQYYKTQGY* |
| Pompano (g) | SEQ ID | FRYAILAREEEPRVRRAALVDKPRVEIADVLISTFTESGVIEVVLQALREIGCNDLRERFAKDTSEGSPTS |
| (Trachinotus | NO: 3 | ASKYGDIMKVETTGASMQTAQQDYLDFSGARASHAMAETDLIEMNNYKSVIKNAAGKKGVDPALIAA |
| ovatus) | | MISRSCRAGKTLSGGWGCWDEKRQKYNTYGLMQIDVNPKGGGHTPKGSWDSEEHLCQAIDILIRFITRI |
| | | RQKYPQWSKEEQLKGGIAAYNAGDGNIGPGKDVDSKTTNGDYANDIVARAQWYKSNGGF* |
| Chlamysin (i) | SEQ ID | AHNFATGIVPQSCLECICKTESGCRAIGCKFDVYSDSCGYFQLKQAYWEDCGRPGGSLTSCADDIHCSS |
| (Chlamys | NO: 4 | QCVQHYMSRYIGHTSCSRTCESYARLHNGGPHGCEHGSTLGYWGHVQGHGC* |
| islandica) | | |

### EXAMPLES

### Example 1: Expression Construct, Transformation, Protein Purification and Processing

Six expression constructs were created for recombinant expression of a mature form of a g-type foodstuff preserving enzyme with SEQ ID NO: 1 (rFPE1) in Pichia pastoris. Constructs included the AOX1, Pex11, DAS1, FLD1, FGH1 and FDH1 promoters. An rFPE1 coding sequence (encoding SEQ ID NO: 1) was fused in-frame with the alpha mating factor signal sequence downstream of the promoter sequences. A transcriptional terminator from the AOX1 gene was placed downstream of the rFPE1 sequence.

A P. pastoris strain was modified to remove cytoplasmic killer plasmids and then further modified to have a deletion in the AOX1 gene. This deletion generated a methanol-utilization slow (mutS) phenotype that reduces the strain's ability to consume methanol as an energy source. P. pastoris cells were transfected with one of the six expression constructs.

Fermentation: Cells transfected with one of the six rFPE1 expression constructs were grown in separate bioreactor at ambient conditions. A seed train for the fermentation process began with the inoculation of shaker flasks with liquid growth broth.

The culture was grown at 30° C., at a set pH and dissolved oxygen (DO). The culture was fed with a carbon source.

To expand production, an rFPE1 P. pastoris seed strain was removed from cryo-storage and thawed to room temperature. Contents of the thawed seed vials were used to inoculate liquid seed culture media in baffled flasks which were grown at 30° C. in shaking incubators. These seed flasks were then transferred and grown in a series of larger and larger seed fermenters (number to vary depending on scale) containing a basal salt media, trace metals, and glucose. Temperature in the seed reactors are controlled at 30° C., pH at 5, and DO at 30%. pH is maintained by feeding ammonia hydroxide which also acts as a nitrogen source. Once sufficient cell mass is reached, the grown rFPE1 P. pastoris is inoculated in a production-scale reactor containing basal salt media, trace metals, and glucose. Like in the seed tanks, the culture is also controlled at 30° C., pH 5 and 30% DO throughout the process. pH is again maintained by feeding ammonia hydroxide. During the initial batch glucose phase, the culture is left to consume all glucose and subsequently-produced ethanol. Once the target cell density is achieved and glucose and ethanol concentrations are confirmed to be zero, the glucose fed-batch growth phase is initiated. In this phase, glucose is fed until the culture reaches a target cell density. Glucose is fed at a limiting rate to prevent ethanol from building up in the presence of non-zero glucose concentrations. In the final induction phase, the culture is co-fed glucose and methanol which induces it to produce rFPE1. Glucose is fed at an amount to produce a desired growth rate, while methanol is fed to maintain the methanol concentration at 1% to ensure that expression is consistently induced. Regular samples are taken throughout the fermentation process for analyses of specific process parameters (e.g., cell density, glucose/methanol concentrations, product titer, and quality). After a designated amount of fermentation time, secreted rFPE1 is collected and transferred for downstream processing.

The rFPE1 products were purified by separating cells from the liquid growth broth, performing multiple filtration steps of the liquid growth broth, performing chromatography, and/or drying the final protein product to produce isolated recombinant rFPE1 powder.

### Example 2: Specific Activity of rFPE1 Versus Chicken Muramidase

An initial suspension concentration of lyophilized Micrococcus luteus (M. lysodeikticus), 0.05% or 5 mg in 10 ml potassium phosphate buffer (KPI 50 mM, pH between 6.2 and 6.6) was prepared and mixed by inversion (30 seconds) to suspend. The solution was allowed to settle for approximately 15-20 min to re-hydrate the cells appropriately. Then the A450 absorbance of cell suspension was determined to be between 0.6-0.7. Before performing the assay, the cell suspension was added to the microplate, the temperature adjusted to 25° C. It was observed that the specific activities of the rFPE1 preparations (from Example 1) were nearly an order of magnitude greater than that of chicken muramidase in this assay as shown in Table 2 below.

TABLE 2
Specific activity results

| | | | Purified chicken |
|---|---|---|---|
| | rFPE1 | Lysovin | muramidase, Sigma |
| Concentration (mg powder/ml) | 1 | 1 | 1 |
| Total Protein (mg/mL) | 0.84 | 0.9 (from product | 0.9 (from product |
| | | description) | description) |
| Shugar Activity, U/mL, microplate | 194,000 ± | 87,487 ± 8,512 | 51,762 ± 1,437 |
| | 18,410 | | |
| Specific Activity U/mg protein | 230,952 ± | 79,533 ± 9,458 | 57,513 ± 1,597 |
| (microplate) | 21,917 | | |
| Range fold increase of rFPE1 | 2.9-4.1 | | |
| specific activity over | | | |
| Lysovin/chicken muramidase | | | |

### Example 3: Xanthan Gum

A functional assay was performed to address whether or not rFPE1 could replace chicken muramidase (Lysovin) in the production of xanthan gum from Xanthanonas campestris. Several enzymatic industrial processes exist for the production of bacterial polysaccharides (including xanthan, gellan and diutan gums) from cell cultures.

Bacterial cultures were heat killed at 55° C. under alkaline conditions (pH 10) then proteolyzed/solubilized using an alkaline protease (a subtilisin-related serine protease). Following proteolysis, preparations were buffered to a neutral pH (e.g., pH 6.5-7.5) and enzymatically treated with muramidase at 25° C. using a concentration of ˜30 ppm (parts per million). For xanthan gum, reactions were terminated by the extraction of solid gum using 1.6× weight of 99% isopropanol. Gum residues were then dried, resuspended in water to a desired final % (w/v) and their qualities assessed by measuring light transmittance through the sample (% T; or clarity at 600 nm). Xanthan gum was produced using the various enzyme concentrations of rFPE1 (from Example 1) or chicken muramidase. Transmittance results are shown in Table 3.

TABLE 3
Transmittance of Xanthan gum preparations

| | | | | | | | | | Control- |
|---|---|---|---|---|---|---|---|---|---|
| Enzyme/ | | | | | | | | | no |
| % Transmittance | 1 ppb | 10 ppb | 100 ppb | 300 ppb | 1 ppm | 3 ppm | 10 ppm | 30 ppm | enzyme |
| EXP#1 | | | | | | | | | |
| (1% sol’s): | | | | | | | | | |
| FPE1 | | | | | | | 89.82 | 85.05 | 54.27 |
| Chicken | | | | | | | 89.76 | 85.05 | 54.27 |
| muramidase | | | | | | | | | |
| EXP#2 | | | | | | | | | |
| (1% sol’s): | | | | | | | | | |
| FPE1 | | | | 80.02 | | 78.85 | | 69.22 | 47.81 |
| Chicken | | | | 78.73 | | 79.48 | | 67.01 | 47.81 |
| muramidase | | | | | | | | | |
| EXP#3 | | | | | | | | | |
| (1% sol’s): | | | | | | | | | |
| FPE1 | 74.98 | 79.75 | 79.44 | | 80.62 | | | | 50.4 |
| Chicken | 45.38 | 58.66 | 81.44 | | 82.04 | | | | 50.4 |
| muramidase | | | | | | | | | |
| EXP#4 | | | | | | | | | |
| (3% sol’s): | | | | | | | | | |
| FPE1 | | | | | | | 89.76 | 85.05 | 54.26 |
| Chicken | | | | | | | 89.82 | 85.05 | 54.26 |
| Muramidase | | | | | | | | | |

### Example 4: Antimicrobial Activity

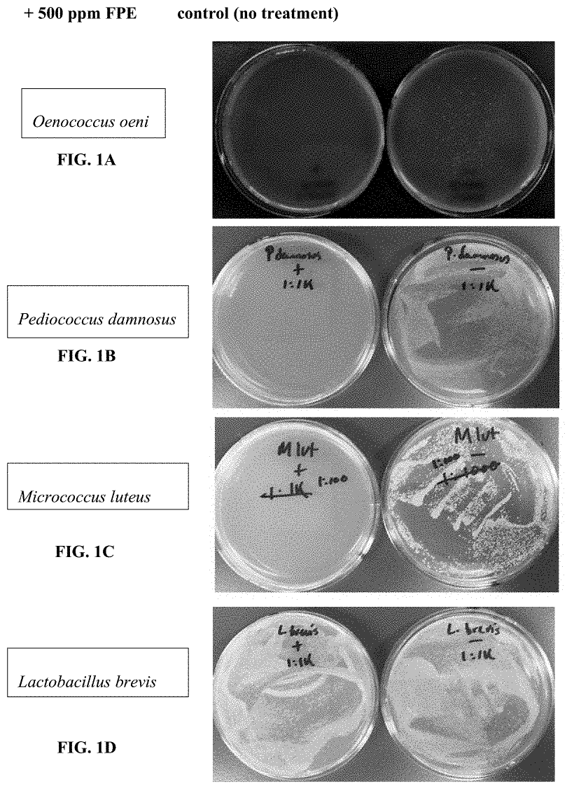

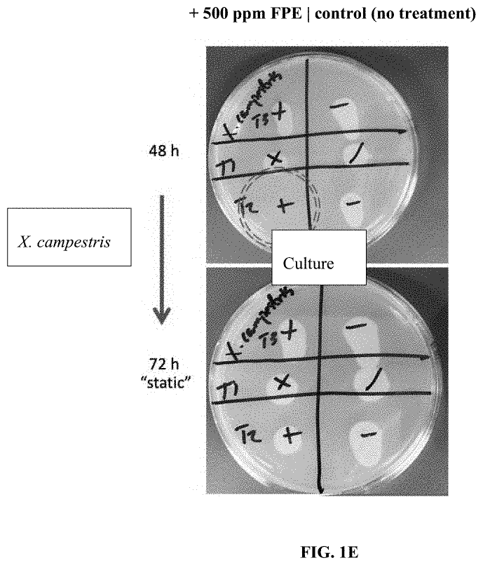

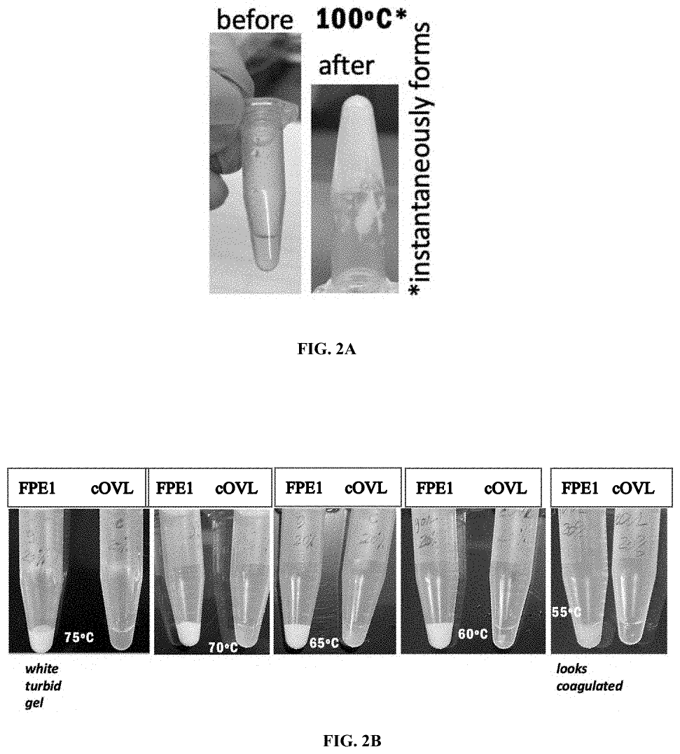

Egg white muramidase, due to its bacterial cell wall degrading activity, is a common preservative/antibacterial added directly to food and beverage products as it is certified Generally Regarded As Safe (GRAS). Therefore, rFPE1 (from Example 1) was tested in a colony forming units (CFU) assay to determine if it could kill live cultures (˜2×10⁷ cells/ml) of Oenococcus oeni (wine, beer and fruit juice contaminant), Pediococcus damnosus (beer and wine spoilage), Micrococcus luteus (food spoilage), Lactobacillus brevis (a beneficial bacterium used in beer making) and Xanthamonas campestris (xanthan gum production). Results of these experiments are shown in FIG. 1A to FIG. 1E.

For this experiment, bacterial cultures were grown in their respective preferred medium and then adjusted to a final OD600 of 1.0 using water. Cell suspensions were then mixed with 500 ppm rFPE1 (final concentration) or water only (control) and allowed to incubate for 1 h at room temperature (25° C.) before cell dilution and plating (see below). Shown in FIG. 1A to FIG. 1E, rFPE1 provided significant bactericidal activity against O. oeni, P. damnosus, and M. luteus cells however rFPE had no visible effect on the viability of a beneficial bacteria, L. brevis. Although rFPE1 is not routinely used to kill live Xanthamonas campestris cells during xanthan gum production, rFPE1 at 500 ppm was demonstrated to control the growth of this bacterial species statistically for up to 48 hrs after treatment (see, FIG. 1E). Thus, these results demonstrate that the bactericidal/bacteriostatic activity of rFPE1 could substitute for any food or beverage application where chicken egg white muramidase is currently used.

In another experiment, the minimum inhibitory concentration of rFPE1 and chicken muramidase enzymes were measured. Results from two different repeats of this assay are shown in Tables 4 and 5 below.

TABLE 4
Minimum inhibitory concentrations in an initial experiment

| | MIC (PPM) | |
|---|---|---|
| | | Commercially |
| | | available chicken |
| Species | FPE1 | muramidase |
| Salmonella enteritidis | >2,000 | >2,000 |
| Listeria monocytogenes | 1,000 | 500-1,000 |
| Staphylococcus epidermidis | >2,000 | >2,000 |
| Pseudomonas aeruginosa | >2,000 | >2,000 |
| Bacillus cereus | >2,000 | >2,000 |
| Clostridium tyrobutyricum | 25 | 50 |
| Lactobacillus plantarum | >2,000 | 2,000 |
| Vibrio parahaemolyticus | >2,000 | >2,000 |
| Aeromonas hydrophila | 25 | 25 |
| Xanthomonas campestris | >2,000 | >2,000 |
| Micrococcus luteus (LB no salt) | 5 | 5 |
| Micrococcus luteus (LB with salt) | 25 | 12.5 |

TABLE 5
Minimum inhibitory concentrations in an subsequent experiment

| | MIC (PPM) | |
|---|---|---|
| | | Commercially |
| | | available chicken |
| Species | FPE1 | muramidase |
| Salmonella enteritidis | >2,000 | >2,000 |
| Listeria monocytogenes | >2,000 | 1,000 |
| Staphylococcus epidermidis | >2,000 | >2,000 |
| Pseudomonas aeruginosa | >2,000 | >2,000 |
| Bacillus cereus | >2,000 | >2,000 |
| Clostridium tyrobutyricum | 50 | 50 |
| Lactobacillus plantarum | >2,000 | >2,000 |
| Vibrio parahaemolyticus | >2,000 | >2,000 |
| Aeromonas hydrophila | 50 | 12.5 |
| Micrococcus luteus (LB no salt) | 5 | 5 |

### Example 5: Gelation of rFPE1

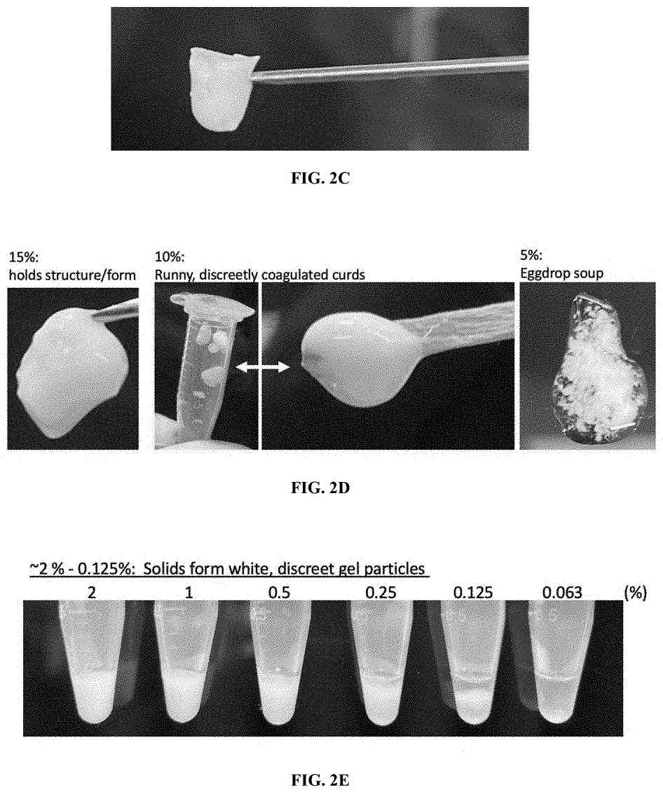

FPE1 with SEQ ID NO: 1 (rFPE1) was produced recombinantly as detailed in Example 1. A 20% rFPE1 solution was made in 1×PBS (pH 7.4). 29 mg rFPE1 powder was resuspended in 145 μl 1×PBS. A tube comprising the solution was dropped into a boiling water bath at 100° C. for a few seconds. FIG. 2A shows the solution before the boiling and after the boiling. As shown in FIG. 2A, rFPE1 formed a gel almost instantaneously.

100 μl solution of 20% rFPE1 solution was heated in tubes at temperatures 55° C., 60° C., 65° C., 70° C., or 75° C. for 10 minutes followed by placement in ice and storage at 4° C. A comparative solution of a recombinant chicken c-type lysozyme (cOVL) at 20% concentration was also heated at the same temperatures. Results are shown in FIG. 2B. As shown in FIG. 2B, rFPE1 gelled at temperatures starting 60° C. whereas cOVL did not gel even at 75° C. FIG. 2C shows the gelled rFPE1 with dents and clean edges.

Gelation of a rFPE1 solution was also measured at different concentrations ranging from 0.063% to 15% upon treatment at 75° C. for 15 minutes followed by storage on ice. rFPE1 was able to gel at concentrations as low as 0.063% where it formed discreet gel particles. At higher concentrations, rFPE1 formed curd-like structures and formed a structured gel particle at the 15% concentration.

Gelation at the temperatures described in this example is relatively unknown in lysozyme proteins and is thus unexpected. These results show that rFPE1 (due to its low thermal gelation profile (glass-transition temperature) may be used as a nucleator for protein gelation and antimicrobial properties in complex protein-protein or protein-carbohydrate food compositions or food products.

### Example 6: Immune Reactivity of rFPE1

Cross-reactivity of antibodies directed against cOVL or rFPE1 were tested on lysovin, on a commercially-available c-type OVL, and on rFPE1. SDS-PAGE was performed for rFPE1, Lysovin, cOVL and a diluted goose egg-white using an anti-cOVL or anti-FPE1 primary antibody. Lanes 1-8 were treated with a 1:3000 dilution of the anti-cOVL antibody whereas lanes 9-16 were treated with a 1:10,000 dilution of an anti-rFPE1 antibody.

TABLE 5
Lanes for SDS-Page

| Lane | Protein |
|---|---|
| 1 | BioRad PrecisionPlus prestained MW markers |
| 2 | BioRad PrecisionPlus prestained MW |
| | markers |
| 3 | empty |
| 4 | 0.01 ug lysovin |
| 5 | 0.1 ug lysovin |
| 6 | 1 ug lysovin |
| 7 | 0.01 ug AKTA purified recombinant rFPEl |
| 8 | 0.1 ug AKTA purified recombinant rFPEl |
| 9 | 1 ug AKTA purified recombinant rFPEl |
| 10 | 0.02 ug cOVL (Sigma L3790) |
| 11 | 0.2 ug cOVL (Sigma L3790) |
| 12 | 2 ug cOVL (Sigma L3790) |
| 13 | 1:4000 dilution of goose egg white |
| 14 | 1:400 dilution of goose egg white |
| 15 | 1:40 dilution of goose egg white |

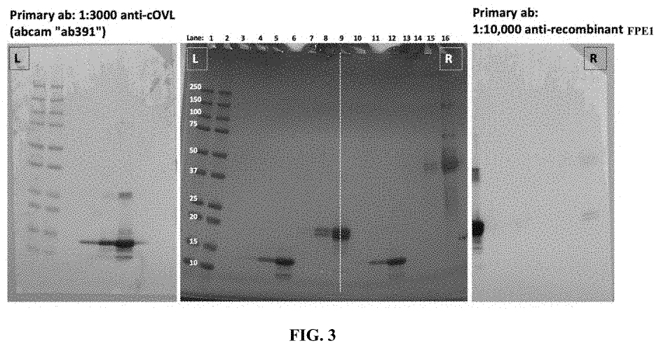

As shown in FIG. 3, the left western blot shows that the anti-cOVL antibody binds to lysovin (MW of about 13 kD). In the right gel, the anti-r rFPE1 antibody binds to purified rFPE1 (MW of about 20 kD). The results show that a commercially available antibody to the chicken egg white lysozyme protein (a known food allergen) does not recognize recombinant or native goose egg white lysozyme.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-66. (canceled)

67. A consumable composition comprising a recombinant food preserving enzyme (rFPE), wherein the FPE is a goose-type FPE (gFPE); wherein the composition is solid, semi-solid or a gel composition.

68. The consumable composition of claim 67, wherein the gFPE comprises an amino acid sequence with at least 95% identity to any one of SEQ ID NOs: 1-4.

69. The consumable composition of claim 67, wherein the consumable composition is heat treated.

70. The consumable composition of claim 67, wherein the consumable composition has a longer shelf life than a nearly identical consumable composition which does not comprise the gFPE.

71. The consumable composition of claim 70, wherein the consumable composition has a longer shelf life than the shelf life of a nearly identical consumable composition which comprises chicken egg-white muramidase rather than the gFPE.

72. The consumable composition of claim 67, wherein the gFPE has an activity of greater than about 90,000 Shugar U/mg wherein one Shugar unit is an amount of the enzyme which will digest a suspension of M. luteus cells causing a decrease in absorbance of the solution at a rate of 0.001 per minute at 37° C., pH 7.0.

73. The consumable composition of claim 67, wherein the gFPE is produced in a yeast cell.

74. The consumable composition of claim 73, wherein the yeast cell is a Pichia cell.

75. A food composition, wherein the food composition comprises one or more consumable ingredients and the gFPE of claim 67.

76. The food composition of claim 75, wherein the food composition has a longer shelf life than a nearly identical food composition which does not comprise the gFPE.

77. The food composition of claim 76, wherein the food composition has a longer shelf life than the shelf life of a nearly identical product which comprises chicken egg-white muramidase rather than the gFPE.

78. The food composition of claim 75, wherein the food composition has at least 0.05% gFPE by weight.

79. The food composition of claim 75, wherein the food composition has at most 10% gFPE by weight.

80. The food composition of claim 75, wherein the food composition is substantially free of microorganisms or bacterial cell-debris.

81. The food composition of claim 75, wherein the gFPE provides favorable qualities of increasing gelling and firmness to a solid or semi-solid food product or of increasing viscosity to a liquid food product, optionally, wherein the food product lacks an additional gelling agent.

82. The consumable composition of claim 67, wherein the consumable composition is a powder.

83. The consumable composition of claim 82, wherein the consumable composition is a food ingredient.

84. A method of preparing a consumable composition comprising steps of:
(a) providing an isolated foodstuff preserving enzyme (FPE) which is recombinantly produced, wherein the FPE is a goose type FPE (gFPE); and
(b) combining the gFPE with one or more consumable ingredients.

85. The method of claim 84, wherein the recombinantly produced gFPE is recombinantly produced in a yeast cell.

86. The method of claim 84, wherein the gFPE has an amino acid sequence with at least 95% sequence identity to one of SEQ ID NOs: 1-4.


SEQUENCE LISTING (4 sequences)

SEQ ID NO: 1 - protein, 185 aa, Anser anser anser [sequence omitted]

SEQ ID NO: 2 - protein, 184 aa, Ambystoma mexicanum
SGCYGNIMDVPTTGASCLTASQDNLPYCGVAASQQMAATDLPDMNQYKEKILAVAQNLCMDGAVIAGIISRESRAGAVLQNGWGDNGHAFGLMQIDIRWHSIEGAWNSQENINEGTGILINMIVAISDKFPSWSVNDNLKGGIAAYNAGPGNIYSYSQVDQYTTDGDYSNDVVARAQYYKTQGY

SEQ ID NO: 3 - protein, 266 aa, Ambystoma mexicanum
FRYAILAREEEPRVRRAALVDKPRVEIADVLISTFTESGVIEVVLQALREIGCNDLRERFAKDTSEGSPTSASKYGDIMKVETTGASMQTAQQDYLDFSGARASHAMAETDLIEMNNYKSVIKNAAGKKGVDPALIAAMISRSCRAGKTLSGGWGCWDEKRQKYNTYGLMQIDVNPKGGGHTPKGSWDSEEHLCQAIDILIRFITRIRQKYPQWSKEEQLKGGIAAYNAGDGNIGPGKDVDSKTTNGDYANDIVARAQWYKSNGGF

SEQ ID NO: 4 - protein, 120 aa, Chlamys islandica
AHNFATGIVPQSCLECICKTESGCRAIGCKFDVYSDSCGYFQLKQAYWEDCGRPGGSLTSCADDIHCSSQCVQHYMSRYIGHTSCSRTCESYARLHNGGPHGCEHGSTLGYWGHVQGHGC